(19) United States
(12) Patent Application Publication
(10) Pub. No.: US 2026/0053992 A1
(43) Pub. Date: Feb. 26, 2026

(54) Oxygenator For Neonates

(71) Applicant: Vitara Biomedical, Inc., Philadelphia, PA (US)

(72) Inventors: Christopher Gregory, Philadelphia, PA (US); Marcus G. Davey, Philadelphia, PA (US); William Gesler, Philadelphia, PA (US)

(21) Appl. No.: 19/110,995

(22) PCT Filed: Sep. 14, 2022
(86) PCT No.: PCT/US2022/043529

§ 371 Date: Mar. 12, 2025

(51) Int. Cl. A61M 1/16; B01D 19/00; B01D 63/02; B01D 71/26

(52) U.S. Cl. CPC A61M 1/1625; A61M 1/1629; B01D 19/0031; B01D 63/033; B01D 71/26; A61M 2205/02; A61M 2240/00; B01D 2313/20; B01D 2313/60

(57) ABSTRACT

Disclosed is a blood oxygenator for a premature fetus comprising a housing defining a cavity therein, wherein the housing comprises a blood inlet, a blood outlet, a gas inlet, and a gas outlet; and a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits, wherein the oxygenator is designed to rely on the fetal heart as sole blood pump.

### BACKGROUND OF THE INVENTION

This disclosure relates to blood oxygenators for use in extracorporeal oxygenation of a patient's blood supply and, in particular, to the extracorporeal oxygenation of a neonate's blood supply in a system designed to provide extracorporeal support for a premature fetus, thereby promoting the growth and enhancing the viability of the fetus.

Blood oxygenators are commonly used to accomplish the gas exchange functions normally performed by the lungs in medical situations when a patient's lungs are temporarily disabled and/or incapable of performing their normal function. Conventional blood oxygenators contain a gas exchange medium, such as a filter membrane made from hollow fibers, across which blood is flowed. The filter membrane is connected to an oxygen supply such that oxygen is diffused from the filter membrane into the blood and carbon dioxide is removed from the blood into the filter membrane. The liquid side boundary layer is the limiting factor in transferring oxygen. The thickness of the boundary layer is generally dependent on the velocity of the flow, the kinematic viscosity of the fluid, and the diameter of the surface.

Oxygenating the blood of a developing fetus has its own unique challenges. For such patients, fetal circulatory regulation of blood flow dynamics is critical, and any oxygenator needs to exhibit at least one or more of the following characteristics: very low resistance, low priming volume, low trans-membrane pressure drops, and efficient gas exchange to ensure optimum fetal physiology.

Accordingly, there is a need in the art for improved blood oxygenators having an increased gas exchange efficiency and a smaller size compared to conventional blood oxygenators for use with neonatal or pediatric patients.

### SUMMARY OF THE INVENTION

In one aspect, the present invention provides a blood oxygenator comprising: a housing defining a cavity therein, wherein the housing comprises a blood inlet, a blood outlet, a gas inlet, and a gas outlet; a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits, wherein blood moves through the oxygenator without actuation from an external mechanical pump.

In another aspect, the present invention provides a system for providing extracorporeal support for a premature fetus, the system comprising: a gas source; a chamber for housing a neonate; an oxygenation circuit comprising: a blood oxygenator comprising: a housing defining a cavity therein, wherein the housing comprises a blood inlet, a blood outlet, a gas inlet in communication with the gas source, and a gas outlet in communication with the gas inlet; and a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits; and a heating element separate from the oxygenator to heat the circuit, wherein the system does not include an external mechanical pump.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a schematic of an exemplary extracorporeal support system according to an aspect of the disclosure.

FIG. 2 illustrates a schematic of a portion of an extracorporeal support system according to another aspect of the disclosure.

FIG. 3 illustrates an isometric view of a portion of an extracorporeal support system according to yet another aspect of the disclosure.

FIG. 4 illustrates an isometric view of an embodiment of an oxygenator according to an aspect of the disclosure.

FIG. 5 illustrates an isometric view of a portion of a gas exchanger according to an aspect of the disclosure.

FIG. 6 illustrates an isometric view of a gas exchanger according to another aspect of the disclosure.

FIG. 7A-7C provide several depicts of another embodiment of an oxygenator according to the disclosure. FIG. 7A provides a conceptual view of blood and oxygen flows relative to a hollow-fiber arrangement. FIG. 7B is a perspective view of an assembled oxygenator housing. FIG. 7C is a perspective exploded view of the oxygenator of FIG. 7B, exposing the fiber stack and components.

### DETAILED DESCRIPTION OF THE INVENTION

All references, including publications, patent applications, and patents, cited herein are hereby incorporated by reference to the same extent as if each reference were individually and specifically indicated to be incorporated by reference and were set forth in its entirety herein.

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about 0.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

As used herein, the term “askew” or “skew” means neither substantially parallel nor substantially right angles (e.g., perpendicular) to a specified or implied line. Skewed intersections of fibers, for example, may range from 0.1 to 179.1 degrees, inclusive of the recited values.

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

As used herein, the terms “perpendicular” or “substantially perpendicular” mean a relative angle as between two objects (if extended to theoretical intersection), such as elongated objects and including reference lines, that is from 88° to 92°, or from 89° to 91°, or from 89.99° to 90.1°, °, inclusive of the recited values.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions or decimals thereof unless the context clearly dictates otherwise).

Method steps described herein may be designated by alphabetical letters, Roman numerals, Arabic numerals, or equivalents thereof. Steps in the method can be performed consecutively, concurrently, or out of order. Further at least a portion of the step can be performed prior to, during, or after another step.

Aspects of the invention provide methods and systems for oxygenating blood. Aspects of the invention can utilize a high flow of CO₂-rich sweep gas. Such aspects avoid the technical challenges of regulating small flow rates of oxygen-rich sweep gas posed by the low neonatal blood flow rate.

### Extracorporeal Support Systems

In embodiments, disclosed is a system for providing extracorporeal support for a premature fetus, the system comprising: a gas source; a chamber for housing a neonate; an oxygenation circuit comprising: a blood oxygenator comprising: a housing defining a cavity therein, wherein the housing comprises a blood inlet, a blood outlet, a gas inlet in communication with the gas source, and a gas outlet in communication with the gas inlet; and a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits; and a heating element 20) separate form the oxygenator to heat the circuit, wherein the system does not include an external mechanical pump.

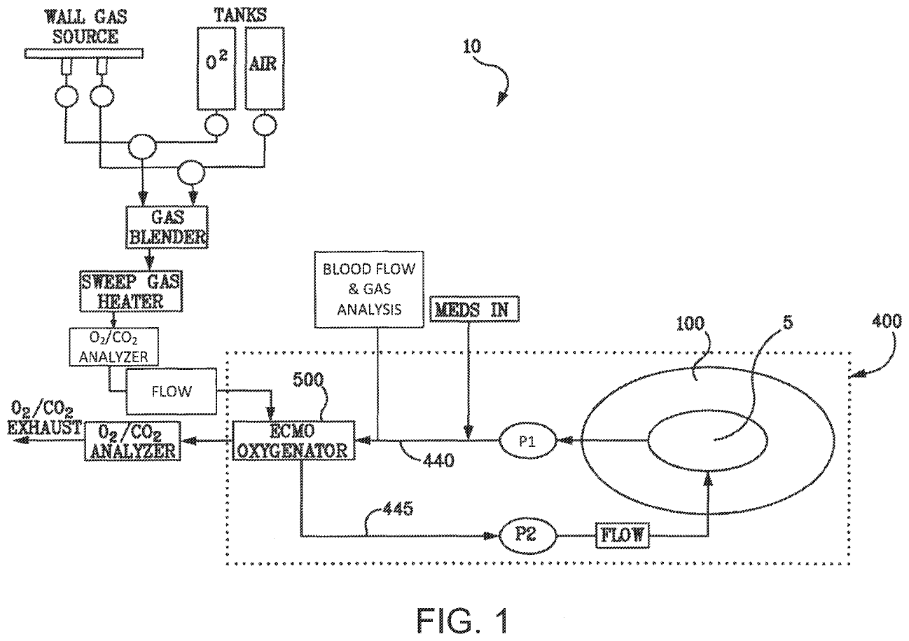

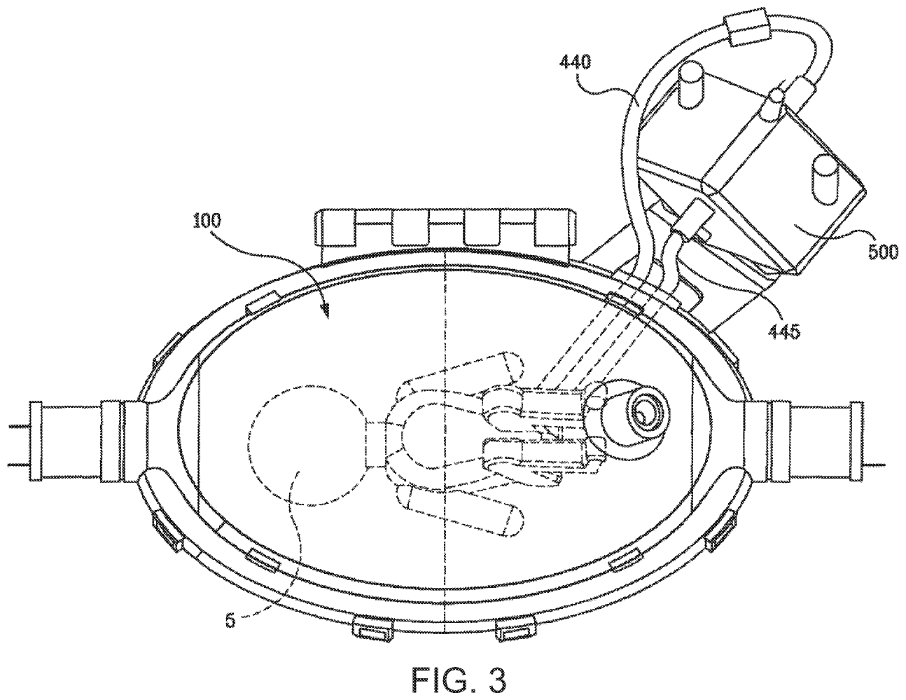

Referring now to FIGS. 1-3, an exemplary extracorporeal support system particularly useful for sustaining life in premature neonates is shown. A system 10 is configured to provide extracorporeal support to a neonate. According to one aspect of the disclosure, the system 10 may be configured to provide a system environment that is similar to an environment the neonate would experience in utero. Viability of a neonate that is removed from the uterine environment (e.g., due to preterm birth) and that is, for example, between about 22 weeks to about 24 weeks gestation, may be increased by placing the neonate in system 10.

According to an aspect of the disclosure, the system environment may be configured to perform at least one or more of the following: (1) limit exposure of the neonate to light; (2) limit exposure of the neonate to sound; (3) maintain the neonate submerged within a liquid environment; (4) maintain the neonate within a desired temperature range; or (5) any combination thereof. The system also permits neonatal activities (e.g., neonatal breathing movements, neonatal swallowing of fluid) necessary for organ growth and development.

The system 10 may be configured to treat neonates (e.g., less than 37 weeks estimated gestational age, particularly 28 to 32 weeks estimated gestational age), and extreme premature neonates (about 23 to 28 weeks estimated gestational age). The gestation periods are provided for humans, though corresponding preterm neonates of other animals may be used. In a particular embodiment, the neonate has no underlying congenital disease. The term or preterm neonate may have limited capacity for pulmonary gas exchange, for example, due to pulmonary hypoplasia or a congenital anomaly affecting lung development, such as congenital diaphragmatic hernia. In a particular aspect, the subject may be a preterm or term neonate awaiting lung transplantation, for example, due to congenital pulmonary disease (e.g., bronchoalveolar dysplasia, surfactant protein B deficiency, and the like). Such transplantation surgeries are currently rarely performed in the United States. However, the number of transplantation surgeries may be increased with the more stable method for pulmonary support provided by the instant invention. The neonate 5 may also be a candidate for ex utero intrapartum treatment (EXIT) delivery, including patients with severe airway lesions and a long-expected course before definitive resection. The neonate 5 may also be a neonatal surgical or fetoscopic procedure patient, particularly with preterm labor precipitating early delivery. According to one aspect of the disclosure, the system 10 may be configured such that the neonate 5 is maintained in the system 10 for as long as needed (for example, for days, weeks or months, until the neonate 5 is capable of life without the system 10). The system 10 should be operable to maintain the neonate 5 for at least 7 days, at least 14 days, at least 21 days, at least 28 days, at least 35 days, at least 42 days, at least 49 days, or at least 56 days.

The system 10 includes a neonatal chamber 100 configured to house a neonate 5, a physiologic saline solution (PSS) circuit configured to provide a flow (e.g., a constant flow) of PSS through the neonatal chamber 100, and an oxygenation circuit 400 configured to remove carbon dioxide from the neonate's blood and supply oxygen to the neonate's blood.

The system 10 is configured to maintain the neonate 5 in the neonatal chamber 100 immersed in PSS. The system 10 is further configured such that the oxygenation circuit 400 provides adequate gas exchange for the neonate 5 to sustain life. In this way, the system 10 provides an environment similar to an intrauterine environment to facilitate continued growth and development of the neonate 5. The system 10 may include a cart or similar device (not shown) that facilitates monitoring, caring for, and transporting the neonate 5 within a medical facility.

According to an aspect of this disclosure, the system 10 may be as described in pending U.S. Patent Application Publication No. 2019/0380900. Another system 20) contemplated for use is described in PCT application number ______ filed Sep. 14, 2022 titled “Systems And Methods For Oxygenating Blood, Passive Oxygenation Circuits, And Neonatal Extracorporeal Support Systems” the disclosure of which is hereby incorporated by reference as if set forth in its entirety herein.

The oxygenation circuit 400 can be connected with the neonate 5 in a venous/venous arrangement. Alternatively, the oxygenation circuit 400 may be connected with the neonate 5 in an arterial/venous arrangement. Cannulas may be placed in the great neck vessels (e.g., carotid, jugular) of the neonate 5 to connect the circulatory system of the neonate 5 to the oxygenator 500. The placement in the great neck vessels may avoid issues of vasospasm and cannula instability in umbilical vessels. An external portion of the cannulas may be fitted with a sleeve (e.g., to permit increased tension of the stabilizing sutures). The sleeve may be made of silicone and may be, for example, about 1 to about 10 cm in length, particularly about 3 to about 5 cm in length. The cannulas may be sutured to the neonate 5 (for example via the fitted sleeve) to secure the cannulas to the neck of the neonate 5.

In some embodiments, the oxygenation circuit 400 may be connected to the neonate 5 via the neonate's umbilical cord. In such an arrangement, cannulas may be sutured into the veins and arteries of the umbilical cord. It will be appreciated that other connection arrangements may be utilized. An exemplary non-suturing device is described in U.S. Patent Application Publication No. 2021/0338270.

The oxygenation circuit 400 may include an oxygenator 500 for providing gas exchange functionality, particularly of oxygen (to) and carbon dioxide (from), to the neonate 5. The oxygenator 500 can be removably connected to the neonate 5 and, optionally, to other components of the oxygenation circuit 400 and the system 10. The oxygenator 500 is connected with the neonate 5 via two or more fluid lines and includes at least a drain line 440 and an inlet line 445. Blood flows from the neonate 5 though the drain line 440 to the oxygenator 500. The blood then flows through the oxygenator 500 and returns to the neonate 5 via the inlet line 445.

In some embodiments, the oxygenator 500 may be configured to be disconnected and replaced while the oxygenation circuit 400 is operational. If the oxygenator 500 is damaged or has surpassed its expected life cycle, the oxygenation circuit 400 may be temporarily configurable to bypass the oxygenator 500 so that the oxygenator 500 may be disconnected from the oxygenation circuit 400 and a new, primed, oxygenator 500 connected in its place without interruption of blood flow.

As will be described in greater detail below, the system disclosed herein is specifically designed to rely on the neonate's heart for blood flow circulation and, accordingly, does not comprise an external mechanical pump, i.e., external to the neonate's heart.

### Blood Oxygenator

In other embodiments, disclosed herein is a blood oxygenator comprising: a housing defining a cavity therein, wherein the housing comprises a blood inlet, a blood outlet, a gas inlet, and a gas outlet; a gas exchanger disposed within the cavity, wherein the gas exchanger comprises a plurality of hollow fibers arranged in such a way as to allow blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits, wherein blood moves through the oxygenator without actuation from an external mechanical pump. A sweep gas typically comprises oxygen, carbon dioxide, and optionally nitrogen.

Referring to FIGS. 1, 4, 7B, and 7C, the oxygenator 500 includes a housing 502 that defines a cavity 540 therein. The housing 502 may include a plurality of ports that extend through the housing 502 into the cavity 540. A blood inlet port 504, at which blood from the neonate 5 can enter the oxygenator 500, is disposed on the housing 502. In some embodiments, multiple blood inlet ports 504 may be configured to receive, either alternatingly or simultaneously, blood from the neonate 5. The blood inlet port 504 is connected to drain line 440, through which the blood moves from the neonate 5 to the oxygenator 500.

In one embodiment, the oxygenator described herein maximizes the pathway of blood flood therethrough to allow contact with the hollow fibers within the housing.

Housing 502 is small enough that, when assembled, the oxygenator 500 can be fully primed with a volume of blood of from about 20 mL to about 85 mL, preferably from about 25 mL to about 55 mL, and preferably from about 30 mL to about 40 mL, and more preferably from about 30 mL to about 35 mL. In some embodiments, the oxygenator disclosed herein has a priming volume of from about 10 mL to 50 mL or from about 20 mL to about 40 mL or from about 20 mL to about 30 mL. In one embodiment, the oxygenator is small enough to be primed with about 30 mL of blood. Such a small priming volume is advantageous because it decreases dilution of the neonate's blood with that of the priming blood. In some embodiments, the oxygenator 500 may have a blood flow range of from about 25 to about 1400 mL/min, from about 25 to about 1000 mL/min, from about 25 to about 750 mL/min, from about 25 to about 500 mL/min, from about 25 to about 200 mL/min, from about 50 to about 175 mL/min, or from about 160 to about 170 mL/min. In one embodiment, the blood flow through the oxygenator 500 is from about 50 mL to about 165 mL/min. The oxygenator 500 may have a gas transfer rate of about 150 mL/min, about 160 mL/min, about 180 mL/min, or greater for oxygen gas (O₂).

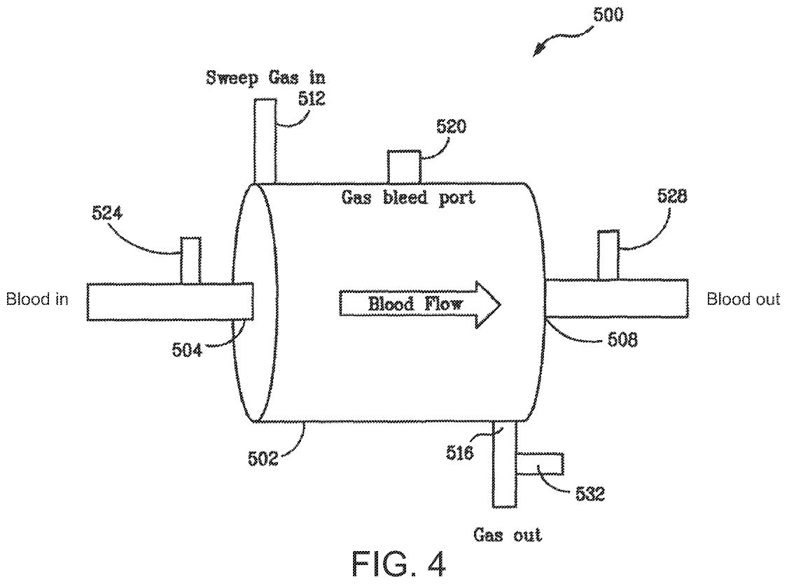

In some embodiments, housing 502 has a window (not shown in FIG. 4) through which the color of the neonatal blood can be seen changing from a maroon color to a bright red color during oxygenation.

One or more additional ports, such as a pressure transducer 524, may be disposed on or adjacent to the blood inlet port 504 or in-line with the drain line 440. The pressure transducer 524 can measure the pressure of the blood from the neonate 5 that enters the oxygenator 500 at the blood inlet port 504. In some embodiments, a sampling port (not shown) may also be disposed on or adjacent to the blood inlet port 504 or the drain line 440 to allow for a portion of the blood entering the oxygenator 500 to be removed from the oxygenation circuit 400 to be analyzed or tested. The sampling port may also be used to inject or infuse medicine or nutrition directly into the blood. The one or more additional ports may have any suitable connection means, such as, for example, a Luer connector or a barb to frictionally hold a plastic line to the port.

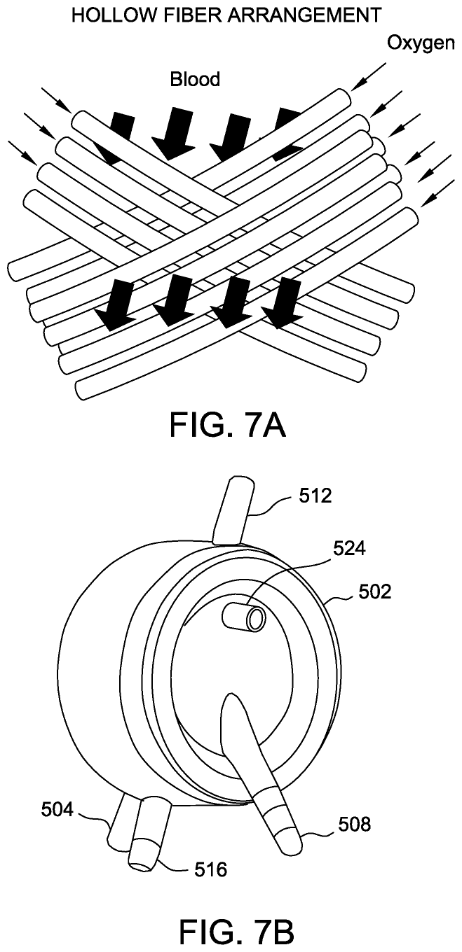

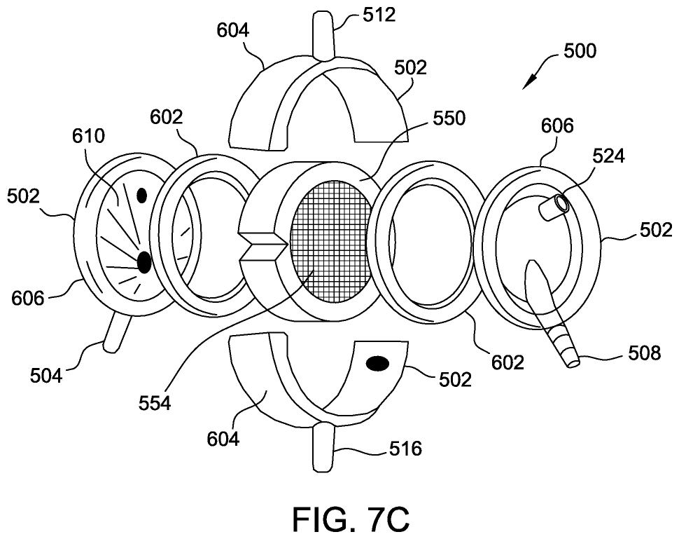

A blood outlet port 508, through which the blood leaves the oxygenator 500 and is returned to the neonate 5, is disposed on the housing 502. The blood outlet port 508 is connected to the inlet line 445, through which the blood moves from the oxygenator 500 to the neonate 5. The number of blood outlet ports 508 may be equal to the number of blood inlet ports 504, or it may be different. As shown in FIG. 4, the blood inlet port/line 504 and blood outlet port/line are on the same axis. In other embodiments such as that shown in FIGS. 7B and 7C, the blood inlet port/line 504 and blood outlet port/line are non-axially symmetric, i.e., they are at angles that are not on the same axis. In a preferred embodiment shown in FIGS. 7B and 7C, the blood outlet port/line is angled downward thereby keeping any air bubbles within the oxygenator. In this or other embodiments, the blood outlet is angled downward relative to the position of the gas inlet. The downward angle of the blood outlet port/line also creates a more uniform blood distribution over the hollow fibers within the oxygenator housing which aids in maximizing gas exchange.

One or more additional ports, such as a pressure transducer 528, may be disposed on or adjacent to the blood outlet port 508 or in-line with the inlet line 445. The pressure transducer 524 can measure the pressure of the blood exiting the oxygenator 500. In some aspects, a sampling port (not shown) may also be disposed on or adjacent to the blood outlet port 508 or the inlet line 445 to allow for a portion of the blood exiting the oxygenator 500 to be removed from the oxygenation circuit 400 to be analyzed or tested.

The sampling port may also be used to inject or infuse medicine or nutrition directly into the blood. The one or more additional ports may have any suitable connection means, such as, for example, a Luer connector.

A fluid flow meter (not shown) may be positioned in-line with the inlet line 445 to monitor the flow rate of the blood returning to the neonate 5 from the oxygenator 500. Typical blood flow rates are from 50 mL/min to 165 mL/min.

A gas inlet port 512 is disposed on the housing 502 for introducing a sweep gas into the oxygenator 500. The sweep gas may include a single gas or a combination of various gases, for example oxygen and other environmental gases. It will be appreciated that the sweep gas may comprise various ratios of gases that may be adjusted to achieve a desired combination and ratio of gases for use with system 10. In some aspects, the sweep gas may have a flow rate of from about 25 milliliter per minute (mL/min) to about 300 mL/min, from about 25 mL/min to about 200 mL/min, from about 50 mL/min to about 175 mL/min, or from about 75 mL/min to about 150 mL/min. In embodiments, the sweep gas flow rate is from about 100 to 200 mL/min. An additional port (not shown) may be disposed on or adjacent to the gas inlet port 512, and a portion of the sweep gas entering the oxygenator 500 may be removed for analysis or testing. The additional port may have any suitable connection means.

In some embodiments, a gaseous medicament can be added to the sweep gas either directly at the source or through an additional port. Such ports can be placed either before or after the blood inlet port 504. One example of a gaseous medicament is nitric oxide, which is used to treat a condition called hypoxic respiratory failure (HRF) where the cells do not receive enough oxygen. Nitric oxide relaxes the blood vessels, so blood flow improves. This allows more oxygen to be picked up by the bloodstream and improves oxygen levels in a neonate's blood. Nitric oxide also decreases platelet adhesion and activation, thus minimizing the risk of clotting. Nitric oxide metabolites can also alleviate umbilical venous spasms. Thus, in another aspect, the present invention provides a method of treating a neonate comprising the steps of connecting a neonate to a system as disclosed herein comprising an oxygenator as disclosed herein; and administering a medicament to the neonate through a sweep gas port in the oxygenator.

A gas exhaust port 516 is disposed on the housing 502 for emitting the sweep gas from the oxygenator 500. An additional port (not shown) may be disposed on or adjacent to the gas exhaust port 516, and a portion of the sweep gas exiting the oxygenator 500 may be removed for analysis or testing. The additional port may have any suitable connection means, such as a Luer connector.

Achieving suitable pressure for both gas and liquid in the oxygenator is preferable. A gas bleed port 520 may be disposed on the housing 502 for removing excess gas when the oxygenator is filled with fluid. Importantly, the pressure of the sweep gas should not be too high, which will cause gas bubbles to bleed into the blood stream which is problematic for the patient. Conversely, blood flow pressures that are too low into the oxygenator 500 may obstruct, slow, or stagnate the blood, which can result in unwanted clotting and/or poor blood circulation for the neonate 5. Unwanted pressure build-up inside the oxygenator 500 may also increase pressure acting on the blood exiting the oxygenator 500 and flowing to the neonate 5. This may increase the flow rate of the blood, which can result in damage to the blood (e.g., to the hemocytes in the blood), leading to unwanted clot formation and decreased blood quality.

In embodiments, the oxygenation circuit 400 is configured such that the blood moves therethrough without actuation from an external pump (e.g., a mechanical pump). Instead, blood is circulated through the drain line 440, the oxygenator 500, the inlet line 445, and any other components by the neonate's heart only. That is, the oxygenation circuit 400 is a passive or pumpless circuit.

As such, it is advantageous to minimize pressures and resistance within the oxygenation circuit 400, and particularly within the oxygenator 500, so that the blood can be moved therethrough without excess obstruction. The use of a pumpless system avoids exposure of the neonate's heart to excess preload encountered in non-pulsatile pump-assisted circuits. The pumpless system also permits intrinsic neonatal circulatory regulation of flow dynamics. The oxygenator 500 preferably has very low resistance, low priming volume, low transmembrane pressure drops, and provides efficient gas exchange. Unwanted pressure build-up in the oxygenator 500, as described above, can also require additional force for moving the blood therethrough. This may put strain on the neonate's heart, leading to health complications. If the heart is unable to overcome the added forces, blood flow may stagnate or slow down significantly, which would lead to stopped or decreased circulation of blood in the neonate.

In some embodiments, the oxygenator 500 exhibits a pressure drop of less than about 50 millimeters mercury (mmHg), less than about 40 mmHg, or less than about 30 mmHg at 1.5 liters/minute (L/min) of blood flow. In other embodiments, the oxygenator 500 exhibits a pressure drop of 5 to 10 mmHg, or from 6 to 9.5 mmHg. The neonatal pressure typically ranges from about 5 mmHg to about 40 mmHg.

Referring to FIGS. 7A and 7C, another embodiment is depicted. As can be seen in FIG. 7C, the oxygenator 500 disclosed herein includes a gas exchanger 550 disposed within a cavity formed by the housing. The blood that enters the cavity at the blood inlet port 504 contacts and flows through and past the gas exchanger 550. The blood then exits the cavity via the blood outlet port 508 on the housing 502. Shown in FIG. 7C is at least one flow guiding feature(s) 610 to assist with blood distribution once the blood flows out of blood inlet 504. Flow guiding feature 610 can be, for example, a vein or a plurality of veins. Yet other embodiments of the flow guiding feature(s) can be a spiral or groove (not shown). The gas exchanger 550 includes a plurality of hollow fibers 554 such that the blood may flow past the fibers 554 while contacting and/or passing close or proximal to the fibers 554. The hollow fibers are microporous and allow at least oxygen and carbon dioxide gases to diffuse between the gas exchanger 550 and the blood flowing through the oxygenator 500. As the blood contacts and/or passes near the fibers 554, diffusion of gases occurs. It will be appreciated that the rate of diffusion may be predetermined and controlled by various aspects, for example, the composition of the sweep gas, the rate of flow of the blood, the rate of flow of sweep gas, the quantity of the fibers 554, the size and shape of the fibers 554, the relative spacing of the fibers 554 within the oxygenator 500, or by other factors that can affect the above variables. Optional spacers 602 are also shown in FIG. 7C and are non-reactive with blood or its components. Preferably, optional spacers are polymeric such as, for example, polyurethane. As shown in FIG. 7C, housing 502 is formed by the assembly of blood caps 606 and gas caps 604, at least one of which may be transparent.

In preferred embodiments, oxygenator 500 is symmetrical in design as is shown in FIGS. 7B and 7C.

Hollow fibers may comprise at least one stack of a fiber mat. Preferably, the fiber surface area ranges from about 0.25 square meter (m²) to about 0.80 m², or from about 0.27 m² to about 0.46 m². The basic form of the oxygenator can be a plurality of fibers such as but not limited to MEMBRANA® (3M Company, St. Paul, Minnesota) PMP (polymethyl pentene). In this or other embodiments, the hollow fibers are comprised of a capillary membrane having at least one or more of the following physical properties: wall thickness ranging from about 80 to about 100 μm; outer fiber diameter ranging from about 350 to about 410 μm; tensile strength ranging from about 150 to about 250 μm; tensile strength at about 60% cN or greater; porosity at about 50% or greater; explosion pressure at about 2.0 bar or greater; and implosion pressure at or about 3.5 bar or greater.

The hollow-fibers materials are contained or “potted” within a housing such as but not limited to a polycarbonate housing (see 550 in FIG. 7C) or other housing materials described herein such that the blood can flow around the outside of the hollow fibers while oxygen flow is provided though the inner lumen of the fibers.

As shown in FIG. 1, two gases, for example an oxygen source and another gas source, can be blended together in a gas blender that blends the oxygen and the air to form the sweep gas. The gas blender can take a variety of forms include a simple wye fitting. Mass flow controller upstream from the gas blender can control the ratio of the gasses. The two gases may be supplied by a high-volume gas reservoir, such as wall lines connected with a central gas supply configured to provide gas to the reservoir.

Alternatively, the two gases maybe supplied from smaller gas reservoirs, such as a portable oxygen tank and a portable air tank. It will be appreciated that a variety of suitable gases may be used. In some embodiments, oxygen and nitrogen gases may be blended to achieve the desired concentration of oxygen. The oxygen concentration may range from 0% to 100% of the blended gas combination.

The hollow fibers 554 may comprise any suitable material such as, for example, polymethylpentene (PMP), polypropylene (PP), polysulfone, polyethersulfone, polyarylethersulfone/polyvinylpyrrolidone, semi-synthetic membrane such as cellulose acetate or cellulose triacetate, a mixture of polyethersulfone (PES) and/or its polymer variants, combined with polyvinylpyrrolidone (PVP), polyacrilonitrile, cellulose triacetate and other cellulosics; PEPA (polyester polymer alloy); and polymethylmethacrylate (PMMA). In preferred embodiments, the hollow fibers 554 comprise polymethylpentene (PMP) due to PMP's desirable qualities of gas permeability. Each fiber 554 may have a receiving end 558, at which the sweep gas can enter the fiber 554, and an emitting end 562, from which the sweep gas exits the fiber 554. A channel 566 extends between the receiving end 558 and the emitting end 562 and is configured to carry the sweep gas through the fiber 554. Hollow fibers 554 as disclosed herein are microporous membranes.

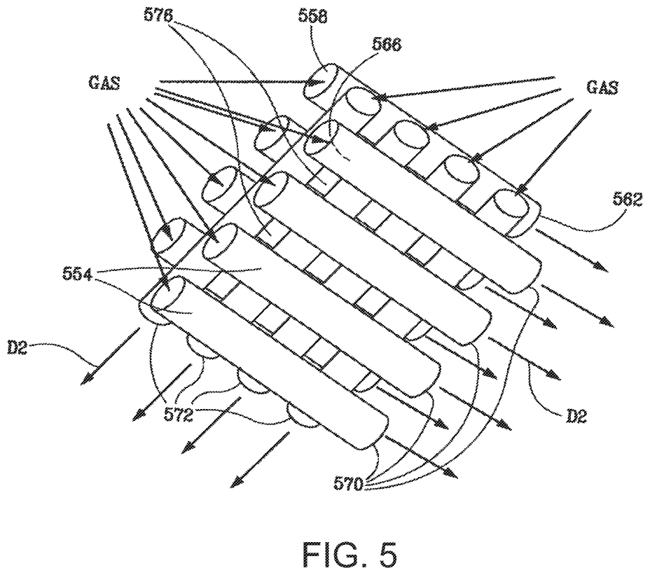

The plurality of fibers 554 may be arranged in a specific or a random pattern to comprise the gas exchanger 550. FIG. 7A illustrates one particular embodiment of a fiber stack suitable for use in the present invention. FIG. 5 illustrates another embodiment.

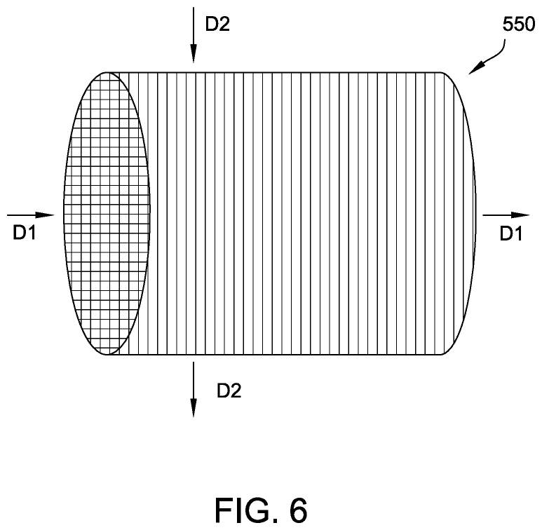

Referring to FIGS. 5-6, in another aspect of the disclosure, the fibers 554 may be arranged in a crisscross pattern or grid. Multiple mats of fibers 554 may be disposed skewed or parallel to each other in a planar arrangement. Preferably, the fiber matts are offset relative to each other as shown in FIG. 7A. Multiple such arrangements may make up the gas exchanger 550, and the orientation of each planar arrangement may be the same as another planar arrangement, the same as all other planer arrangements, or different from other planar arrangements. Referring again to FIGS. 5-6, an exemplary portion of a gas exchanger 550 is shown having a first plane 570 that contains a plurality of fibers 554 and a second plane 572 adjacent to the first plane 570. The planes 570 and 572 are substantially the same, except that the fibers 554 of the second plane 572 are perpendicular to the fibers 554 of the first plane 570. While the second plane 572 is shown to be rotated 90 degrees relative to the first plane, it will be appreciated that other relative angles between adjacent planes of fibers may be utilized. Any suitable number of planes 570, 572 may be arranged to form the gas exchanger 550.

The fibers 554 can be arranged such that a space 576 exists between adjacent fibers to allow the blood to flow through. The size of the space 576 may depend on the quantity and/or density of fibers, the flow rate of the blood, the flow rate of the sweep gas, the desired resistance within the oxygenator, or on other parameters that can affect gas exchange of the blood.

The gas exchanger 550 may include various shapes and configurations, such as cylindrical or cuboidal. Referring to FIG. 6, an arrangement of adjacent planes (e.g., plane 570 and plane 572 located at a 90-degree angle to plane 570) may be arranged as a cylinder having two opposing planar ends. Blood can enter the gas exchanger 550 at one of the planar ends, travel through the gas exchanger 550, and exit at the opposite planar end. Sweep gas can enter the gas exchanger 550 at the curved wall of the cylinder at one location and exit at another location on the curved wall.

Each of the fibers 554 may extend between the top and bottom opposing planar ends of the gas exchanger 550, such that all of the fibers 554 are disposed in an askew or angled positions relative to each other such as is shown, for example, in FIG. 7A. In such an arrangement, the direction of flow of the sweep gas is preferably opposite the direction of flow of the blood. As shown in FIGS. 7B and 7C, for example, the sweep gas inlet 512 may be disposed at one opposing planar end of the oxygenator 500 (e.g., the top end shown in the figure) with the sweep gas exhaust 516 being disposed at the other opposing planar end of the cylinder (e.g., the bottom end shown in the figure), such that the sweep gas flows in a direction from the top to the bottom of the cylinder. The blood inlet port 504 can be arranged at the bottom end shown in the figure, and the blood outlet port 508 can be arranged at the top end shown in the figure, opposite the bottom end, such that the blood flows from the inlet to the outlet and opposite the flow of the sweep gas. This is advantageous because it allows for better and more efficient gas exchange between the blood and the gas-exchange fibers 554.

In some embodiments as shown in FIG. 5, it may be advantageous to arrange the gas exchanger 550 to have a plurality of planes 570, 572 such that they form a cylinder as described above. Such a gas-exchanger would have a circular cross-section perpendicular to the blood flow direction. The circular cross-section eliminates corners, thus decreasing areas of higher turbulent flow and stagnant flows and helps maintain a more even (e.g., constant flow) throughout the gas exchanger 550. This reduces the likelihood of damage to the blood cells and decreases the potential for clot formation. Such an arrangement may be advantageous because it also decreases pressure within the oxygenator 500 and reduces resistance to flow. As noted above, due to the pumpless/passive nature of the system 10, it is important to have as low resistance to the blood flow as possible to allow the neonatal heart to pump blood through the oxygenation 20) circuit 400 without stopping or significantly slowing the flow and without overexerting itself (blood flow can be non-invasively restricted if necessary to avoid starving neonatal blood circulation). Importantly, there should be no folds or wrinkles in the fiber layers, which could potentially cause clotting.

In some embodiments, any portion of the oxygenator that comes into contact with blood, such as without limitation, the gas exchanger 550, the housing 502, the fibers 554, or any of the ports (e.g., blood inlet port or blood outlet port), and tubing (lines) disclosed herein, may be coated or lined with one or more anti-clotting, antithrombogenic, and/or non-thrombogenic materials, such as, but not limited to, immobilized polypeptide, heparin, and phosphorylcholine. The coating for the oxygenator exhibits at least one or more of the attributes: reduces adhesion of fibrinogen and platelets, reduces platelet activation, and/or reduced clot formation. In one embodiment, at least one surface that is exposed to neonate blood is coated with one or more anti-clotting, antithrombogenic, and/or non-thrombogenic materials. In one embodiment, the coating is comprised of Corline Heparin Conjugate (CHC™) provided by Corline Systems AB. In an alternative embodiment, the coating is comprised of phosphorylcholine (PC) polymer coating product provided by Vertullus. Coatings can be applied as recommended by the manufacture such as by spray coating, spin coating, dip coating or similar means.

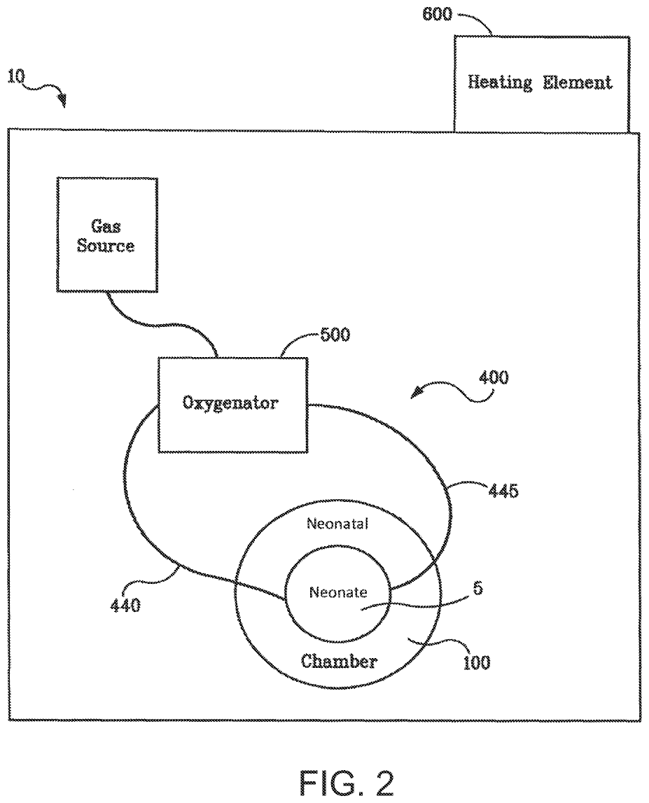

Referring to FIG. 2, the system 10 may include a heating element 600 positioned therein and configured to heat the oxygenation circuit 400; however, the oxygenator itself does not include an integrated heating element. The heating element 600 may heat and maintain a desired temperature of the neonate 5, the environment in which the neonate 5 resides, the enclosure of the oxygenation circuit 400, and other components of the system 10. Still referring to FIG. 2, an exemplary arrangement is depicted in which the heating element 600 is located separate from the oxygenator 500 and contacts the oxygenation circuit 400. FIG. 2 is an exemplary schematic showing an aspect of such an arrangement, and it will be appreciated that the heating element 600 may be disposed elsewhere and may be either directly adjacent or in indirect contact with the oxygenation circuit 400.

By maintaining the entire oxygenation circuit 400 within the desired temperature, there is no need to additionally heat the blood specifically as it flows to, through, and away from the oxygenator 500. As such, a heating element 600 is neither needed nor desired within the oxygenator 500. Excluding the heating element 600 from the oxygenator 500 allows the oxygenator 500 to be smaller, require fewer fibers 554, impose less blood-flow resistance, and require a smaller amount of priming material to operate. It is important to note that an oxygenator within an extracorporeal circuit generally requires a heating element to maintain the desired temperature of the blood traveling therethrough. Failure to do this may result in damage to the blood, shock to the patient, or other health hazards. In the systems described throughout this application, the above drawbacks are eliminated by heating the entire system 10, or at least the oxygenation circuit 400, with the heating element 600. This allows exclusion of a heater from the oxygenator 500 itself, while maintaining the required temperature of the blood and sweep gas moving between the neonate 5 and the oxygenator 500.

As noted above, removing the otherwise-necessary heater from the oxygenator 500 allows for a smaller gas exchanger 550 and a smaller cavity, which in turn allows for a smaller necessary priming volume to operate the oxygenator 500. To start the oxygenation process, the oxygenator 500 must be filled with a suitable priming material. The larger the oxygenator 500, the greater the required minimum volume of priming material. In some embodiments, when a neonate 5 is connected with the oxygenation circuit 400, the priming material comprises adult human blood (e.g., maternal blood or blood from a blood bank). Adult blood has different properties from neonatal blood, and it is preferred to minimize the impact of these differences. Priming the oxygenator 500 with adult blood results in hemodilution of the blood inside the neonate (i.e., the neonatal blood will mix with the adult blood used for priming). The greater the volume of the priming material, the greater the hemodilution. It may be advantageous to minimize the hemodilution within the 20) neonate 5. By excluding a heater from the oxygenator 500 (in lieu of the heating element 600 within the system 10 or the oxygenation circuit 400), the total volume of the oxygenator 500 is decreased, thus requiring a smaller priming volume.

Further, decreasing the total size and volume of the oxygenator 500 also decreases the transit time of the blood as it moves through the oxygenator 500. Increased transit time may lead to thrombosis and clot formation and decreasing the size of the oxygenator 500 decreases the transit time of the blood flowing therethrough, reducing the chance of clot formation. The blood flow rate through the oxygenator 500 may depend on the age and size of the neonate 5. For example, in some embodiments, a neonate weighing approximately 500 grams would have a flow rate of between about 40 mL/min and 60 mL/min. In other embodiments, a 24-week-old neonate may have a flow rate of between about 60 mL/min and about 90 mL/min. The flow rate may be higher in a more developed and larger neonate and will depend, in part, on the weight of the neonate. Suitable flow rates may range between about 75 ml/kg/min and about 175 mL/kg/min.

In one embodiment, the oxygenator disclosed herein has a clinical use of fourteen (14) days or greater or 16 days or greater or 20 days or greater until a replacement oxygenator is needed. Transmit time through the oxygenator has been optimized—much faster (priming volume and circuit flow) quicker blood flow through the oxygenator the less time blood spends within the oxygenator.

Another aspect of the invention provides systems and methods for capturing CO₂ for use in producing CO₂-rich sweep gas. In one embodiment, a CO₂ separator 1014 (e.g., a CO₂-selective membrane such as a glassy polymeric membrane, metal-organic framework (MOF), zeolitic-imidazolate framework (ZIF), and the like, a cryogenic distillation device, and the like) is employed between the oxygenator exhaust and the CO₂ input of the gas blender 1002 and the separated CO₂ is recycled as an input to the gas blender 1002.

### Examples

### Term Definition

O₂ Molecular Oxygen
CO₂ Carbon Dioxide
N₂ Molecular Nitrogen
Hb Hemoglobin
SO₂ or Blood oxygen saturation (Percent of Hb molecules bound to O2) SAT
PO₂Partial pressure of Oxygen
PCO₂ Partial pressure of Carbon Dioxide
BE or the difference between the observed and the normal buffer base
Base concentration
Excess
α Solubility of O₂ in plasma (0.00314 mL O₂/(mmHg PO₂*dL blood))
β Oxygen binding capacity of hemoglobin (1.34 mL O₂/g Hb)
Q Volumetric flow rate, Qb (blood), Qg (gas)
Sweep the gas flowing to and through the oxygenator
Effluent the gas exiting the oxygenator (Sweep gas composition changes in the
Gas Oxygenator due to exchange with blood, what exits the oxygenator is the effluent gas)
P Pressure
ΔP change in pressure between two points
PMP Poly Methyl Pentene or Material of Hollow fibers in the tested oxygenator
CoO2 Inlet O₂ Content
VO2 Volume of O₂ transferred between blood and gas circuit
VCO2 Volume of CO₂ transferred between blood and gas circuit
I Subscript used to denote oxygenator inlet
O Subscript used to denote oxygenator outlet
PC Phosphorylcholine polymer coating provided by Vertellus in tested oxygenator
Rated Flow rate at which the Oxygenator exit saturation reaches 95% under
Flow standard inlet conditions.

### Device Tested

A blood oxygenator as disclosed herein was evaluated for its performance with various coatings. The device tested was composed of hollow PMP fibers oriented in consecutive offset or askew layers and cut into a circular shape. The circular stack (cylinder) was enclosed along the circumference with a polyurethane housing and enclosed at each end with polycarbonate blood caps with barbed connectors forming a fluid tight blood path where blood enters on one side, flows over the outside of the hollow fibers, and exits on the other side of the oxygenator. The outer side of the polyurethane housing was cut to expose the hollow fiber herein and enclosed with polycarbonate end caps to form a gas pathway where sweep gas enters the oxygenator, flows through the hollow fiber lumens contained therein, and exits the oxygenator on the other side.

By way of diffusion, gas transfer occurs across the PMP hollow fibers. In normal operation, low O₂, high CO₂ blood enters the oxygenator and contacts the outer fiber walls while high O₂ low CO₂ sweep gas enters the oxygenator and contacts the inside fiber wall. The concentration gradient across the fiber walls drives diffusion of O₂ into the blood (quantified as VO₂) and removal of CO₂ from the blood (quantified as VCO₂).

Test parameters were controlled to be equivalent across the different oxygenator test configurations. In general, testing was done in conformance with the ISO 7199:2016 standard with minor modifications that are more relevant to the system described herein and fetal physiology (specifically, Inlet saturation level, Inlet pCO₂ level, and gas to blood flow ratios). Additionally, this testing was not run for 6 hours per device as three exemplary devices were tested in the same test day and blood pool.

This test generates a comparison of baseline gas exchange function (VO₂ and VCO₂) of 3 configurations of the oxygenator: Corline-coated or Heparin-based coating, Vertellus-coated or PC polymer coating, and uncoated.

### Samples

Device A—uncoated
Device B—Vertellus PC or phosphorylcholine (PC) polymer coating
Device C—Corline or heparin coating

### Procedure

Pressure transducers were zeroed to atmospheric pressure before use and blood flow meters were set to the correct fluid (blood at 37 C) and zeroed with the pump off.

Blood Supply: 4 liters (L) of donor bovine blood (drawn by venipuncture) was provided. Blood was collected in CPDA-1 solution and collected in 1 L IV bags. Blood was drawn and shipped the day prior to testing in an insulated container with ice packs for temperature maintenance at 2-8° C. Blood was cold upon arrival.

Blood Conditioning: Upon arrival blood was filtered through a 40 μm transfusion filter, heparinized (4500 Units/L), and dosed with antibiotics (100 mg gentamicin/L blood).

Saline was removed from the circuit and blood was added and conditioned to the following parameter ranges:
Inlet Saturation: 40+/−5%*
*Deviation from 7199 standard to better match the intended use environment of the oxygenator (fetal physiology). Standard is 65+/−5%.
Hemoglobin: 12+/−1 g/dL
Base Excess: 0+/−5 mmol/L
pCO2: 42+/−7 mmHg**
** Deviation from ISO 7199 standard to better match the intended use environment of the oxygenator (fetal physiology). Standard is 45+/−5 mmHg.
PH: 7.4+/−0.1
Temperature 37+/−2 C

### Data Collection

3 blood flows were tested per device and 3 gas flows were tested per blood flow set point per the table below.

| Device | Qb (mL/min) | Qg:Qb |
|---|---|---|
| A | 50 | 1:1, 2:1, 3:1 |
| (uncoated) | 100 | 1:1, 2:1, 3:1 |
| | 165 | 1:1, 2:1, 3:1 |
| B | 50 | 1:1, 2:1, 3:1 |
| (PC coating) | 100 | 1:1, 2:1, 3:1 |
| | 165 | 1:1, 2:1, 3:1 |
| C | 50 | 1:1, 2:1, 3:1 |
| (Heparin coating) | 100 | 1:1, 2:1, 3:1 |
| | 165 | 1:1, 2:1, 3:1 |

*Deviation from ISO 7199 standards to approximate anticipated use scenario of the device. Standard calls for 0.5:1, 1:1, and 2:1 gas to blood flow ratios. However, 0.5:1 gas to blood flow ratios will not be used clinically in the system, 3:1 was chosen as a more appropriate ratio.

At each condition Qb, P1, P2, QgO2, FeCO2, and ΔPg were recorded from the appropriate meters. When inlet saturations reached the target range a pre and post oxygenator blood sample was pulled and measured in the blood gas analyzer. The SO₂, Hb, pO₂, pCO₂, PH, and BE parameters for each device were recorded. From these parameters, VO₂, VCO₂, and pressure drop are calculated per RD-P-0042.

The PC coated device or Device B was tested last and after the data points listed above were collected (primary) an additional data set under standard ISO 7199 inlet conditions was gathered (secondary). Inlet conditions were set to:
Inlet Saturation: 65+/−5%
Hemoglobin: 12+/−1 g/dL
Base Excess: 0+/−5 mmol/L
pCO2: 45+/−5 mmHg
pH: 7.4+/−0.1
Temperature 37+/−2 C

Gas flow rate was set to 1:1 ratio of blood flow rate and blood flow rates were set to 50, 100, and 165 mL/min and then additional flow rates were tested until rated flow was determined (exit saturation dropped below 95%). Due to limitations of the gas blender, sweep flow was capped at 500 mL/min so a 1:1 gas to blood flow ratio was not achievable at the higher flow rates.

Finally, post oxygenator samples were taken from the oxygenator pigtail and the blood circuit to compare after the last data points were collected and the circuit was deoxygenating slowly.

### Observed Results

VO₂ as function of blood flow: minimal differences observed between coated and uncoated configurations.

VCO₂ as a function of blood flow with 1:1 gas to blood flow ratio. VCO₂ is highest in the uncoated device, followed by the PC coated device, and then by the Heparin coated device.

VCO₂ as a function of blood flow with 2:1 gas to blood flow ratio. VCO₂ is highest in the uncoated device, followed by the PC coated device, and then by the Heparin coated device.

VCO₂ as a function of blood flow with 3:1 gas to blood flow ratio. VCO₂ is highest in the uncoated device, followed by the PC coated device, and then by the Heparin coated device.

Thus, oxygen transfer appears relatively unaffected by device coating (at least when 100% O₂ is used as the sweep gas). All configurations were able to fully oxygenate the incoming blood from 40% to 100% saturation. Pressure drop also appeared to be unaffected by device coating.

Regarding CO₂, both coatings (PC and Heparin) reduced carbon dioxide transfer rate. The PC coating reduced VCO₂ by an average of 40% relative to the uncoated device. The Heparin coating reduced VCO₂ by an average of 61% relative to the uncoated device.

A follow up study was performed to examine using higher sweep gas flow rates with the PC coated device to increase VCO₂. The same test set up described above was used except that additional gas flow meters capable of higher sweep gas measurements were added (EQ-0012 & EQ-0047). The same blood flow rates were tested (50, 100, and 165 mL/min) but higher gas flow rates were used (300-3000 mL/min).

The data showed that VCO₂ is sweep dependent in the sweep flow rate range tested (300-3000 mL/min). The data matches a natural log curve well, thus showing VCO₂ increases with increasing sweep in the range tested.

Although preferred embodiments of the invention have been described using specific terms, such description is for illustrative purposes only, and it is to be understood that changes and variations may be made without departing from the spirit or scope of the following claims.

We claim:

1. A blood oxygenator comprising:
a housing defining a cavity therein, wherein the housing comprises a blood inlet, a blood outlet, a gas inlet, and a gas outlet; and
a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits, wherein blood moves through the oxygenator without actuation from an external mechanical pump.

2. The blood oxygenator of claim 1 wherein the housing further comprises at least one pressure transducer.

3. The blood oxygenator of claim 1 wherein the housing further comprises at least one port to inject a substance into the blood or withdraw a sample of blood.

4. The blood oxygenator of claim 1 wherein the housing further comprises at least one gas bleed port.

5. The blood oxygenator of claim 1 wherein the oxygenator exhibits a pressure drop of about 50 mmHg or less.

6. The blood oxygenator of claim 5 wherein the oxygenator exhibits a pressure drop of less than about 40 mmHg.

7. The blood oxygenator of claim 6 wherein the oxygenator exhibits a pressure drop of about 10 mmHg or less.

8. The blood oxygenator of claim 1 wherein the oxygenator has a priming volume of about 50 mL or less.

9. The blood oxygenator of claim 8 wherein the oxygenator has a priming volume of about 40 mL or less.

10. The blood oxygenator of claim 9 wherein the oxygenator has a priming volume of about 30 mL or less.

11. The blood oxygenator of claim 1 wherein the oxygenator has a priming volume of between about 20 mL and about 50 mL.

12. The blood oxygenator of claim 11 wherein the oxygenator has a priming volume of between about 30 mL and about 40 mL.

13. The blood oxygenator of claim 1 wherein the oxygenator has a blood flow range of up to about 2.0 L/min.

14. The blood oxygenator of claim 1 wherein the oxygenator has a blood flow range of up to about 2.5 L/min.

15. The blood oxygenator of claim 1 wherein the oxygenator has a blood flow range of up to about 2.8 L/min.

16. The blood oxygenator of claim 1 wherein the oxygenator has a gas transfer rate of about 150 mL/min or greater for O₂.

17. The blood oxygenator of claim 16 wherein the oxygenator has a gas transfer rate of about 160 mL/min or greater for O₂.

18. The blood oxygenator of claim 17 wherein the oxygenator has a gas transfer rate of about 180 mL/min or greater for O₂.

19. The blood oxygenator of claim 1 wherein the hollow fibers comprise polymethylpentene (PMP).

20. The blood oxygenator of claim 1 wherein the blood inlet is located at a bottom end of the oxygenator and the blood outlet is located at a top end of the oxygenator; and the gas inlet is located at the top end of the oxygenator and the gas outlet is located at the bottom end of the oxygenator, such that blood and sweep gas flow in opposite directions.

21. The blood oxygenator of claim 1 wherein the hollow fibers are arranged in a crisscross pattern.

22. The blood oxygenator of claim 21 wherein the crisscross patterns comprise multiple planes of hollow fibers, wherein each plane comprises hollow fibers arranged in parallel, and each plane is stacked such that the hollow fibers of one plane are perpendicular to the hollow fibers of an adjacent plane of hollow fibers, wherein the hollow fibers of each plane are spaced apart to allow blood to flow therethrough.

23. The blood oxygenator of claim 1 wherein the oxygenator has a cylindrical shape.

24. The blood oxygenator of claim 1 wherein the oxygenator comprises at least one internal surface coated with at least one material selected from the group consisting of an anti-clotting agent, an antithrombogenic agent, and a non-thrombogenic agent.

25. The blood oxygenator of claim 24 wherein the at least one material is an immobilized polypeptide or heparin.

26. The blood oxygenator of claim 1 wherein the housing comprises a window through which one can see the blood being oxygenated.

27. A system for providing extracorporeal support for a premature fetus, the system comprising:
a gas source;
a chamber for housing a neonate;
an oxygenation circuit comprising:
a blood oxygenator comprising:
a housing defining a cavity therein, wherein the housing comprises a blood inlet, a blood outlet, a gas inlet in communication with the gas source, and a gas outlet in communication with the gas inlet; and
a gas exchanger disposed within the cavity, wherein the gas exchanger comprises a plurality of hollow fibers arranged in a pattern allowing blood to flow past the hollow fibers when in contact with the hollow fibers, wherein the hollow fibers comprise a receiving end in communication with the gas inlet for receiving a sweep gas and an emitting end in communication with the gas outlet from which the sweep gas exits; and
a heating element separate form oxygenator to heat the circuit, wherein the system does not include an external mechanical pump.

28. The system of claim 27 wherein the housing further comprises at least one pressure transducer.

29. The system of claim 27 wherein the housing further comprises at least one port to inject a substance into the blood or withdraw a sample of blood.

30. The system of claim 27 wherein the housing further comprises at least one gas bleed port.

31. The system of claim 27 wherein the oxygenator exhibits a pressure drop of less than about 50 mmHg.

32. The system of claim 5 wherein the oxygenator exhibits a pressure drop of less than about 40 mmHg.

33. The system of claim 6 wherein the oxygenator exhibits a pressure drop of less than about 30 mmHg.

34. The system of claim 27 wherein the oxygenator has a priming volume of less than about 50 mL.

35. The system of claim 34 wherein the oxygenator has a priming volume of less than about 40 mL.

36. The system of claim 35 wherein the oxygenator has a priming volume of less than about 30 mL.

37. The system of claim 27 wherein the oxygenator has a priming volume of between about 20 mL and about 50 mL.

38. The system of claim 37 wherein the oxygenator has a priming volume of between about 30 mL and about 40 mL.

39. The system of claim 27 wherein the oxygenator has a blood flow range of up to about 2.0 L/min.

40. The system of claim 27 wherein the oxygenator has a blood flow range of up to about 2.5 L/min.

41. The system of claim 27 wherein the oxygenator has a blood flow range of up to about 2.8 L/min.

42. The system of claim 27 wherein the oxygenator has a gas transfer rate of about 150 mL/min or greater for O₂.

43. The system of claim 42 wherein the oxygenator has a gas transfer rate of about 160 mL/min or greater for O₂.

44. The system of claim 43 wherein the oxygenator has a gas transfer rate of about 180 mL/min or greater for O₂.

45. The system of claim 27 wherein the hollow fibers comprise polymethylpentene (PMP).

46. The system of claim 27 wherein the blood inlet is located at a bottom end of the oxygenator and the blood outlet is located at a top end of the oxygenator; and the gas inlet is located at the top end of the oxygenator and the gas outlet is located at the bottom end of the oxygenator, such that blood and sweep gas flow in opposite directions.

47. The system of claim 27 wherein the hollow fibers are arranged in a crisscross pattern.

48. The system of claim 47 wherein the crisscross patterns comprise multiple planes of hollow fibers, wherein each plane comprises hollow fibers arranged in parallel, and each plane is stacked such that the hollow fibers of one plane are perpendicular to the hollow fibers of an adjacent plane of hollow fibers, wherein the hollow fibers of each plane are spaced apart to allow blood to flow therethrough.

49. The system of claim 27 wherein the oxygenator has a cylindrical shape.

50. The system of claim 27 wherein the oxygenator comprises at least one internal surface coated with at least one material selected from the group consisting of an anti-clotting agent, an antithrombogenic agent, and a non-thrombogenic agent.

51. The system of claim 50 wherein the at least one material is an immobilized polypeptide or heparin.

52. The system of claim 27 wherein the housing comprises a window through which one can see the blood being oxygenated.

53. The blood oxygenator of claim 1 wherein the blood inlet and the blood outlet are non-axially symmetric.

54. The blood oxygenator of claim 53 wherein the blood outlet is angled downward relative to the position of the gas inlet.

55. The system of claim 27 wherein the blood inlet and the blood outlet are non-axially symmetric.

56. The system of claim 55 wherein the blood outlet is angled downward relative to the position of the gas inlet.
